(19) United States
(12) Patent Application Publication
(10) Pub. No.: US 2003/0065318 A1
(43) Pub. Date: Apr. 3, 2003

(54) Method and tool for epicardial ablation around pulmonary vein

(76) Inventor: Rajesh Pendekanti, Bridgewater, NJ (US)

Correspondence Address: NUTTER MCCLENNEN & FISH LLP, WORLD TRADE CENTER WEST, 155 SEAPORT BOULEVARD, BOSTON, MA, 02210-2604, US

(21) Appl. No.: 09/966,331

(22) Filed: Sep. 28, 2001

(51) Int. Cl. A61B 18/14

(52) U.S. Cl. 606/41

(57) ABSTRACT

A method and device for treating atrial arrhythmia forms a conduction block along a circumferentially oriented path in myocardial tissue circumscribing one or more pulmonary veins at their juncture with the wall of the atrium to transect electrical conductivity of the vein and block conduction between the longitudinal portion of the vein wall and the wall of the left atrium. The method treats a patient with a focal arrythmogenic origin in the pulmonary vein, either ablating the focal origin or isolating the focal origin with an epicardially-formed circumferential conduction block. An ablation device includes a curved or curveable lasso or hook, or a curved tip with an epicardial ablation mechanism that forms small diameter conduction blocks, preferably isolating individual pulmonary veins or a pair of veins while avoiding stenotic sequelae. The lesion sets advantageously reduce the central connecting mass of myocardial tissue at the ostia so it does not sustain a re-entrant signal.

### FIELD OF THE INVENTION

The present invention relates to cardiac ablation and to devices and systems for performing cardiac ablation. It also relates to treating atrial arrythmias, e.g., atrial fibrillation.

### BACKGROUND OF THE INVENTION

Atrial fibrillation is a commonly occurring disorder characterized by erratic beating of the atrium, a condition that may result in thrombogenesis and stroke. While medication can be effective for some cases, many patients are not responsive to medical therapies, and effective treatment of those resistant cases may call for creating lesions in the atrium to form effective conduction blocks.

It is well documented that atrial fibrillation, either alone or as a consequence of other cardiac disease, is the most common cardiac arrhythmia. According to recent estimates, more than one million people in the United States suffer from this arrhythmia, amounting to roughly 0.15% to 1.0% of the population. Moreover, the prevalence of atrial fibrillation increases with age, affecting nearly 8% to 17% of those over 60 years of age.

Although atrial fibrillation may occur alone, it often associates with other cardiovascular conditions, including congestive heart failure, hypertensive cardiovascular disease, myocardial infarction, rheumatic heart disease and stroke. The condition itself presents three separate detrimental sequelae: (1) a change in the ventricular response, including the onset of an irregular ventricular rhythm and an increase in ventricular rate; (2) detrimental hemodynamic consequences resulting from loss of atrio-ventricular synchrony, decreased ventricular filling time, and possible atrio-ventricular valve regurgitation; and (3) an increased likelihood of sustaining a thromboembolic event because of the loss of effective contraction and resulting atrial stasis of blood in the left atrium. Thus, this condition requires treatment.

Atrial fibrillation may be focal in nature, caused by the rapid and repetitive firing of one or more isolated centers within the atrial myocardium. The foci may act as a trigger of the fibrillation, or the foci may sustain the fibrillation. Recent studies have suggested that focal arrhythmias often originate from an ectopic myocardial tissue region within the pulmonary veins extending from the left atrium, and even more particularly in the superior pulmonary veins.

Several approaches to ablating these foci, or to blocking conduction pathways from such foci have been proposed. U.S. Pat. No. 6,012,457 (Lesh) is directed to a device and method for forming a circumferential conduction block in a pulmonary vein. This patent discloses a percutaneous translumenal catheter ablation technique. A limitation of the method disclosed therein is that, as an endocardial approach, it requires access to the endocardial surface.

U.S. Pat. No. 6,161,543 (Cox) is directed to methods of epicardial ablation for creating a lesion around the pulmonary veins. This patent discloses a method for creating a blocking lesion by forming a unitary ablation lesion along a path encircling the pulmonary trunk (e.g., all four pulmonary veins). However, not only can the geometry and access to form a unitary lesion around the pulmonary trunk be quite challenging, but the atrial mass enclosed by the unitary ablation can constitute a sufficiently large region to sustain re-entrant wavefronts within it, so that it is possible that stimuli for fibrillation remain present, or that new arrythmias will arise.

When performing ablation to create conduction blocks, the locations of lesions and their depth determine their effectiveness against particular re-entrant signal paths. Thus, a preliminary mapping step is usually necessary to identify suitable ablation sites, and a follow-up mapping may be required to assure that the created lesions were of sufficient depth. The requirements of sequential mapping of signal patterns and ablation of the required lesions to destroy sites or block pathways, dictates that a cardiac treatment procedure be relatively lengthy. Often, once a lesion is created in a chamber wall, the chamber must be remapped to assure that block has occurred and that the tissue is not simply stunned. The follow-up mapping may need to be performed after waiting a number of minutes, and it may also be found necessary to re-ablate a lesion if the initial treatment lesion did not extend deeply enough to be effective, or was inaccurately placed or otherwise ineffective.

For the described atrial ablations, a number of factors pose impediments to effective ablation, including the generally difficult routes of access to the atrial chamber (or the posterior position of the target vessels in the case of epicardial access), and the requirement for sequential mapping, ablation and visualization steps to confirm effectiveness of the electrophysiological intervention.

There thus remains a need for methods and devices that form ablation lesion sets effective to treat atrial fibrillation.

### SUMMARY OF THE INVENTION

The present invention includes a method for treating atrial arrhythmias by forming a conduction blocking lesion along a circumferential path through myocardial tissue at the juncture of the atrium with a pulmonary vein. The path circumscribes the entry to the pulmonary vein lumen and transects the electrical conductivity of the pulmonary vein relative to its longitudinal axis. Rather than creating a unitary ablation lesion encircling the pulmonary trunk, the method of the invention creates transmural lesions encircling individual pulmonary veins or several veins. Individually, an ablation lesion may include a focal origin or it may be located between a focus and the left atrial wall. The totality of lesions forming a lesion set may reduce the atrial mass of the ostia so that it is too small to sustain a re-entrant wavefront.

An ablation tool of the invention for carrying out the cardiac ablation method includes an ablation head configured to epicardially access the outer surface of the left atrium and which is effective to grasp a pulmonary vein and ablate a circumferential lesion in the root or junction region thereof. The ablation tool includes a tip assembly with a curved, substantially looped or hook-shaped ablation element located on the distal end portion of an elongate probe body. Suitable tip structures include a lasso-type ablation structure that deforms into a substantially circumferential or even closed loop; an opposed-jaw clamping ablation structure that grips the vein about a substantially circumferential contact segment or segments; or a hook ablation element with an end curved on a small diameter that subtends a major arc segment, e.g., preferably from about 90 degrees of arc to about 180 degrees of arc or more. The tip may be curveable, rather than curved, and may have an active ablation length over about one centimeter, and preferably about two to about six centimeters in length. The circumferential ablation element may employ any of a number of different ablation modalities, e.g., cryogenic ablation, RF ablation, ultrasound or the like.

A method according to the invention includes: accessing the outside of the heart (either by open thoracotomy or thoracoscopically); positioning a circumferential ablation element around the outside of a pulmonary vein, moving the ablation element down the vein to a juncture with the atrial wall, and actuating the ablation element to form a circumferential ablation lesion. The lesion forms a circumferential blocking line through myocardial tissue effective to block conduction of signals between the lumen of the pulmonary vein and the atrial wall. By ablating a transmural lesion in myocardial, rather than venous tissue, conduction from the vessel is blocked, while problems of post-ablation stenosis are avoided.

In a further embodiment of one aspect of the invention, the circumferential ablation element includes a member with a working length whose curvature can be adjusted from a straight contour to a curved/circular contour effective to engage the ablation element with the outer surface of the pulmonary vein. The circumferential ablation element is used to form the circumferential lesion such that the width of the lesion (transverse to the longitudinal axis of the pulmonary vein) is shorter than the lesion circumference that circumscribes the junction with the pulmonary vein. The lesion extends entirely through the wall, forming a conduction block that isolates arrythmia signals from any focal origin that may be present in the targeted pulmonary vein.

### BRIEF DESCRIPTION OF THE DRAWINGS

These and other features of the invention will be understood from the description and claims herein, taken together with illustrative drawings, wherein

FIG. 1A schematically illustrates a heart and associated vessels from a front view;

FIG. 1B schematically illustrates a heart and associated vessels from a rear perspective view;

FIG. 1C is a realistic rendition of the heart and associated vessels from the perspective of FIG. 1B;

FIG. 1D illustrates a cutaway perspective view within the left auricle, of the atrium and four pulmonary vein orifices;

FIG. 2 illustrates a perspective view of a first embodiment of an ablation tool of the present invention;

FIG. 3 illustrates a perspective view of a second embodiment of an ablation tool of the present invention;

FIG. 4 illustrates a perspective view of a third embodiment of an ablation tool of the present invention; and

FIG. 5 schematically illustrates one lesion set in accordance with the present invention for treatment of atrial arrythmia.

### DETAILED DESCRIPTION OF THE INVENTION

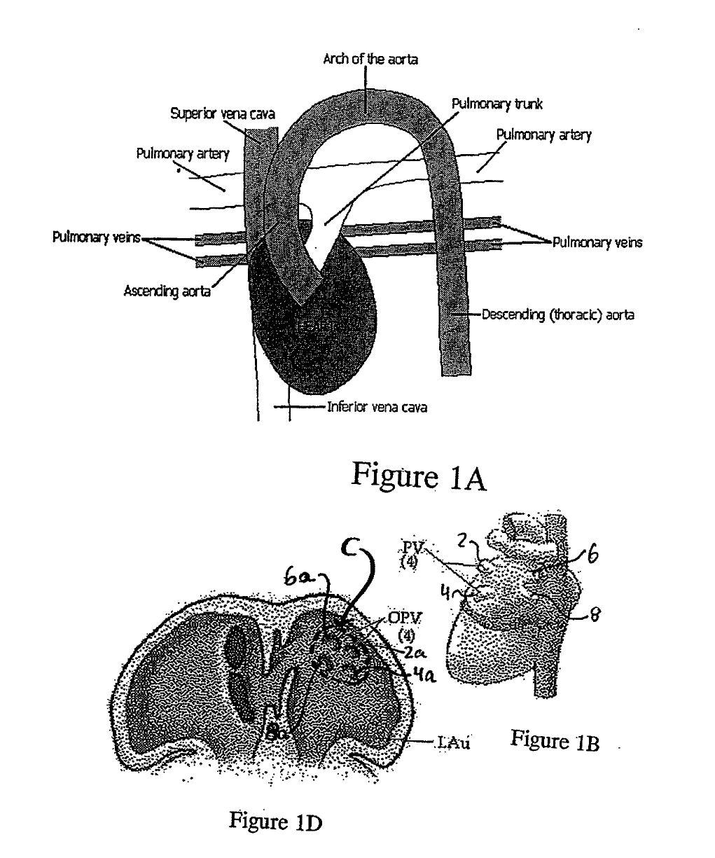

FIG. 1A illustrates a schematic view of a human heart showing major blood vessels, as seen from a frontal aspect. As shown, the four pulmonary veins bearing oxygenated blood from the left and right lobes of the lung connect to the back of the heart, at the left atrium, in a position that is occluded by the heart and aorta (from the front) and by the lung itself (from the back). The actual geometry is better understood from a posterior rendering of the heart, as shown in a stippled schematization in FIG. 1B, in which the superior and inferior pulmonary veins 2,4 from the left lobe and the superior and inferior pulmonary veins 6, 8 from the right lobe of the lungs all enter the left atrium (denoted generally “LA” in FIG. 1C) along a generally horizontal direction from the respective left and right sides at the back of the heart. FIG. 1C shows a more realistic rendition, indicating the relative sizes of vessels, scale of related structures, and the overall features and variability of shapes and tissue texture of the epicardial surface. FIG. 1D shows a view corresponding to FIG. 1B, from the interior of the left auricle, identifying the orifices 2a, 4a, 6a, 8a of the four pulmonary veins.

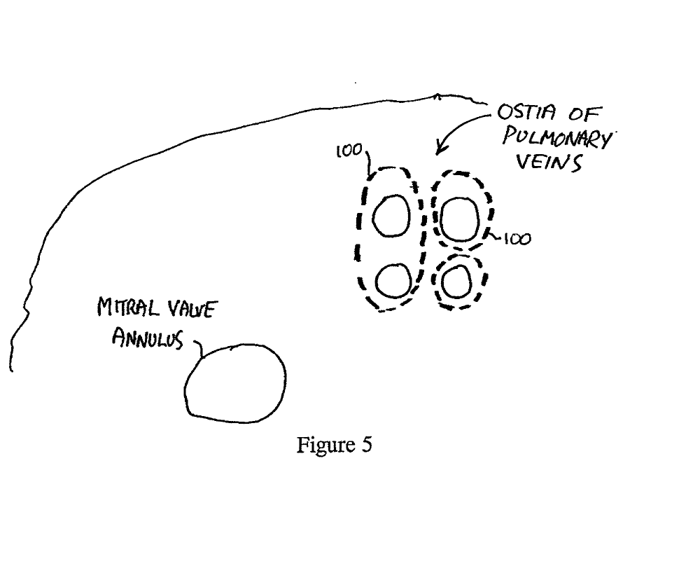

As shown, the atrial wall forms a large continuous region of complex topology in the region of the pulmonary veins, with atrial tissue extending as short cylindrical ingrowths at the opening into each of the four pulmonary veins, and with the four stubby cylinders of myocardial tissue being interconnected by a central continuous connecting region of myocardial tissue (interior to the contour denoted “C” in FIG. 1D) extending between the respective openings. In accordance with a principal aspect of the present invention, a blocking ablation is carried out to prevent arrythmia by epicardially forming one or more encircling lesions (100, FIG. 5) in myocardial tissue at the junction of the vein(s) and the atrium, and each lesion also isolates at least one pulmonary vein from this myocardial tissue connecting region. Since each encircling lesion borders, or extends at least partly along, this central interconnecting region of the ostia, the lesion set also reduces the conductive area and extent of the ostia. In a typical procedure, a further blocking line may be placed endocardially by other means, e.g., by surgically accessing or by endovascularly accessing the interior of the atrium to ablate a blocking line between the ostia and the annulus of the mitral valve. The PV blocking lesions, however, are created by epicardial ablation contact, using an ablation tool configured to access the venous junction and ablate circumferential blocks in myocardial tissue. The device may encircle or grasp a pulmonary vein and be manipulated down to the juncture region of the vein and the atrial wall.

Embodiments of the invention will be described below for a thoracoscopic or minimally-invasive surgery, during which each pulmonary vein isolation lesion is formed using a one or more step process to completely encircle and isolate the vein. Ablation tools of the invention may have either a substantially closable loop end, or an arc, hook, or curved clamp that includes the active ablation element for effecting the required lesions. When the lesions are formed with arc segments using several successive ablation steps, the individual lesions are positioned contiguously, so that there is continuity between the lesion segments. These all overlap and interconnect to form one unitary ablation, extending fully transmurally to the inner surface and transecting conduction paths to the pulmonary vein.

In one method of the present invention, a circumferential conduction blocking lesion is formed around each of the four pulmonary veins (PVs). In other embodiments of the invention, a blocking lesion set is constructed to isolate a pair of PV's together (e.g., forming a loop that encircles the opening to two pulmonary veins), or to isolate three PVs together as a set and the remaining (fourth) pulmonary vein with a separate circumferential lesion. In this manner, by employing plural separate blocking lesions around the pulmonary vein openings at their junction, the isolating lesions each impinge on the central interconnecting atrial mass C (FIG. 1D), reducing its size to a level that prevents this region from sustaining a re-entrant signal wavefront.

In the practice of the invention, when a preliminary mapping reveals that one or more of the four pulmonary veins does not harbor any focal lesions that could trigger atrial fibrillation (or when the physician does not want to ablate one or more of the pulmonary veins for another reason, such as an elevated risk of stenosis), the lesion sets of the present invention may be limited to ablation of the remaining pulmonary veins.

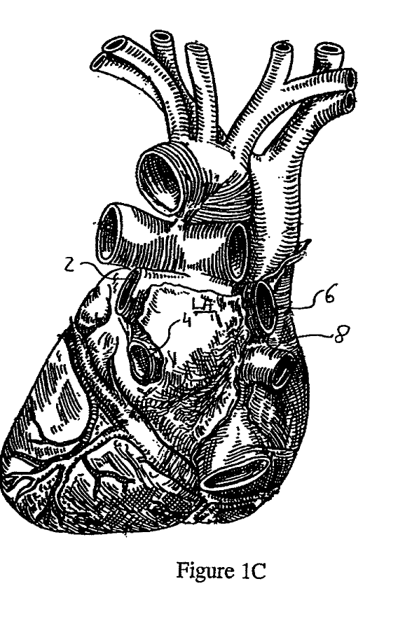

One ablation device of the present invention is a hook probe, schematically illustrated by probe 10 of FIG. 2. The probe 10 has a handle 12, an elongated body 14 and a curved or hooked distal tip section 20 that forms the tissue-contacting active ablation element. The distal tip section 20 can be rigid and of a relatively small curvature, and formed in a generally C-shaped or J-shaped curvature. Alternatively, it may be flexible or malleable, including a suitable mechanism so that its shape may be adjusted to fit a small curvature, e.g., conforming to the outer diameter of a pulmonary vein. The body 14 may also be shapeable (malleable) so that it can be manually bent to a contour that enables it to reach (e.g., through an intercostal portal located at a fixed lateral or posteriolateral position) to a targeted pulmonary vein. Various polymers or polymer-covered metal rods or wires may be used to impart a suitable degree of malleability to allow the body 14 to be repeatedly and reversibly shaped for successively accessing different cardiac sites. The body 14 is preferably sufficiently rigid to allow the tip section 20 to be firmly urged against the targeted tissue region by pressure on the handle.

In use, the distal end including the ablation element is shaped to at least partially encircle a pulmonary vein and it is positioned around the pulmonary vein close to or at the juncture of the vein and the atrial wall. The tip is then energized or otherwise activated to perform an ablation. In an exemplary embodiment, the probe includes a cryogenic delivery mechanism such that the tip forms the lesion by freezing the tissue contacted by the probe. The cryogenic delivery system may include electrically actuated solid state cooling elements, or it may operate by circulation of a coolant or heat exchange medium through the tip, in a manner known in the art. Although freezing is one exemplary method of ablating tissue, probes of the invention may alternatively be fabricated with operative lesion forming energy sources of other, generally known types, such as RF ablation, ultrasound, microwave, laser, or localized delivery of chemical or biological agents, light-activated agents, laser ablation or resistance heating ablation. In general, the fluid lines, electrical conductors, light pipes or other ablation energy or coolant conductors, as well as temperature or other feedback sensors from the probe tip (not shown), will be understood to interface with the probe handle and extend through the body to the tip, in a manner known in the art. The connections at the handle may also include various operator controls, and the energy or coolant and feedback sensor lines may connect to a separate console, e.g., including control circuitry and instrumentation for setting ablation regimens and monitoring the energy delivery.

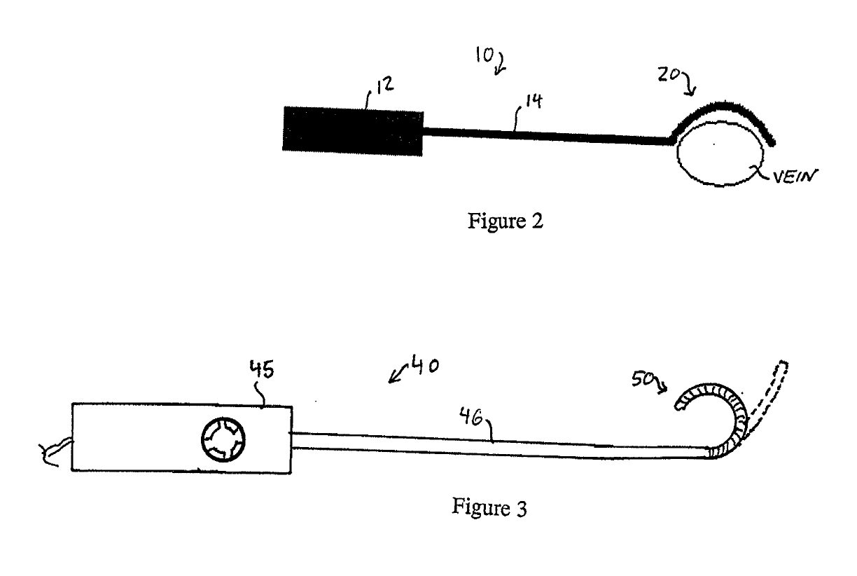

Another ablation probe device for implementing the blocking lesion sets of the present invention is a lasso ablation probe, of which one embodiment is shown in FIG. 3. This embodiment of an ablation device may be similar in overall construction to a conventional hand-held probe device with a multi-axis movable ablation tip, but is preferably constructed such that the movement mechanism for steering or shaping the ablation tip has a range effective to curl the tip about a relatively small diameter conforming to the pulmonary vein circumference. As with the embodiment of FIG. 2, the ablation probe 40 in accordance with this aspect of the invention has a control handle 45 connected via an elongated body 46 to a steerable distal tip 50. The distal tip 50 includes the ablation element, and as shown the distal tip section is deformable to a tightly curled radius such that it grips around the pulmonary vein and is positioned to form the above-described ablation lesions in the myocardial tissue at the junction of vein and the atrium. One suitable mechanism for effecting deformation of the tip is a four-wire multi-axis steering system, for example as described in U.S. Pat. No. 6,123,699. However, numerous other constructions may be used. For example, the probe may be constructed such that the probe tip curls in a single plane (e.g., using one or two control wires, optionally with a compressible tip mounting member), and/or may be constructed such that the angle of the tip plane is varied by an articulation or joint just below the tip, controlled by a separate wire, rod or other control mechanism. Alternatively, the handle may be rotated about its axis to adjust a plane of curvature better conform with the contour of, or seat against, the target lesion area. Indeed, if it is not desired to urge the lasso down against atrial wall tissue but rather to ablate only radially through the myocardial tissue lining the proximal pulmonary vein wall, the loop plane need not be precisely controlled, so long as loop can catch the vessel and the lesion is placed close to the junction of the vessel with the atrial wall.

The lasso probe may be a cryogenic probe, or may be constructed, as described above, using any other ablation modality such as RF ablation, ultrasound, microwave, laser, localized delivery of chemical or biological agents, light-activated agents, laser ablation or resistance heating ablation.

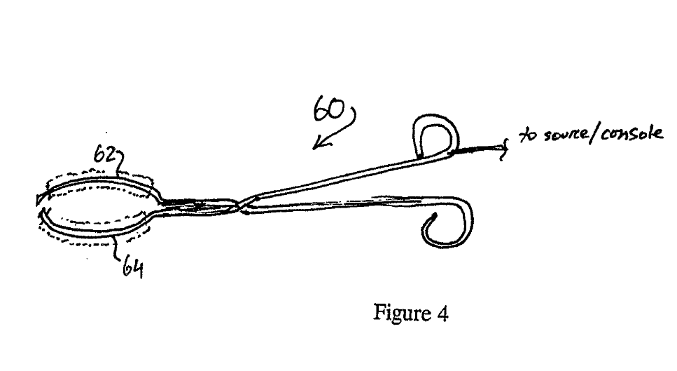

Another ablation probe device for implementing the blocking lesion sets of the present invention is a “test tube holder” ablation probe, i.e., a pliers-, scissor- or forceps-shaped clamping probe, of which one embodiment 60 is shown in FIG. 4. This embodiment of an ablation device is configured as a clamping or grasping device with opposed jaws 62, 64 that, in the illustrated embodiment are concavely curved. The probe opens and closes in a scissoring motion, much like a set of tongs, pliers or forceps, to grasp the juncture region of a pulmonary vein about the curved surface contact area. The concavity of the jaws assures that sufficient contact pressure is exerted over a substantial and continuous segment or pairs of segments of the intended circumferential arc, without inflicting unduly high or destructive crushing pressure on the ostium of the vein. In use, once the tissue is loosely gripped, the physician slides the distal end of the probe down along the vein toward the atrium (if the vessel is gripped), or outwardly toward the vessel (if the ostia is gripped), or so that it is positioned at the junction of the pulmonary vein and the atrial wall. The distal end jaw portion includes an ablation mechanism, indicated in phantom in FIG. 4, which is then energized (e.g., by suitable controls in the handle or in a separate control unit, not shown) to form a circumferential lesion through the wall. As with the above described embodiments, the ablation mechanism may operate using a variety of energy sources or ablation modalities, including a cryogenic element that forms the lesion by freezing the heart tissue, or another ablation modality such as RF ablation, ultrasound, microwave, laser, localized delivery of chemical or biological agents, light-activated agents, laser ablation or resistance heating ablation.

In a representative endoscopic or so-called minimally invasive procedure performed on a beating heart, the patient is sedated, and the physician places a trocar intercostally to create a port for a thoracoscopic approach. Typically a second port is formed (e.g., abdominally) for insufflating the chest cavity and introducing a camera for endoscopic visualization. One or more additional ports may be made for introducing further tools or manipulators. The ablation device is then introduced through the first port. The heart may be rotated to better expose the atrial region. The physician determines the target area where a lesion is to be made, dissects away epicardial fat (if necessary) or resects down toward the trunk of the pulmonary vein with suitable endoscopic tools, and proceeds endoscopically to carry out targeting, positioning and ablation steps on the selected vein or veins. Specifically, the body 14 of the probe 10 (or body 46 of probe 40) is shaped, if necessary, to reach the desired site at the epicardial surface, and the tip is contoured to wrap about the vessel, sliding down to the trunk region or juncture with the cardiac wall, or is contoured to contact that site with the desired arcuate shape. The ablation tip is then actuated with suitable heat-, RF-, light-energy or cooling parameters for a time effective to ablate an arc-like lesion through the wall in myocardial tissue near the trunk of the vein. For example, for a bipolar RF ablation modality, the tip may be actuated with a signal between 10 kHz and several hundred kHz, at a power of about thirty watts for about thirty seconds to form a blocking lesion through the wall. The precise power settings may be varied, based on probable thickness of the wall tissue and lesion or electrode size, and sensor feedback. The ablation step may be carried out in several iterations to form a circumferential blocking lesion. That is, the tool is removed and the shape of the shaft and/or the tip are readjusted to a contour suitable to reach an adjacent position along the intended blocking line, the tool is re-inserted through the portal, and the ablation tip is brought into contact with the target region on the vessel/heart wall, and the ablation tip is re-energized to form a further portion of the circumferential lesion at the target site. The repositioning/electrode actuation sequence is repeated one or more times until a full circumferential blocking lesion has been created, and until all lesions of the set are formed (if more than one blocking lesion is to be made). The ablation tip may also include one or more mechanisms, such as a suction, compression or adhesion mechanism, to better grip the adjacent tissue and assure stable contact during the ablation procedure. Alternatively, various forms of retractor mechanisms and stabilizing feet may be employed, in addition to the probe itself, to immobilize the targeted region of tissue.

It will be understood that the described lesion sets may also be effected during an open chest surgical procedure, using the same or a similar ablation tool. In such case direct visible access to the pulmonary veins at their juncture with the epicardial surface will generally be possible, and the heart is more readily rotated, and positioned or made accessible by a suitable set of surgical retractors. In this case, the target region may be stabilized by a clamping foot adjacent to the region of the desired ablation segment. For open chest procedures, the ablation tool may have a rigid body 14, 46, or several ablation probes may be provided with bodies 14, 46 that connect to the ablation tip portion at different angles or offsets, to provide effective contact reaching to and around the juncture of the pulmonary veins.

A surgical procedure for effecting the described ablation with an open chest and with the heart stopped, but still closed, is performed by sedating and anaesthetizing the patient. A median sternotomy or thoracotomy is performed to gain access to the heart, and the heart is arrested. The major vessels are connected to a heart-lung machine, placing the patient on cardiopulmonary bypass (CPB). The target area for creating lesions is determined on the epicardial surface, and epicardial fat is dissected away as required from the target region. The heart may be rotated and the target region positioned with suitable retractors and an ablation probe 10, 40 or 60 is selected to form the lesion set. The body 20 of the ablation device 10 may be shaped, or an appropriate rigid body provided in a probe 40, suitable for positioning the ablation tip at least partially around one or more pulmonary veins, and down against the region of the juncture. Epicardial tissue may be resected back in that region to position the ablation element close to the juncture on the target site. The ablation element (e.g., electrode set) is then energized (e.g., for about thirty seconds at a thirty Watt power setting) to form a lesion at the target site. The ablation tip is then re-positioned and the electrodes actuated again, with this sequence repeated one or more additional times until a full blocking lesion set has been created.

Such an open chest procedure may be performed ancillary to the performance of another cardiac procedure (e.g., in connection with a mitral valve replacement procedure), to treat preexisting history of atrial fibrillation or, as a prophylactic measure to prevent atrial fibrillation from developing. Alternatively, the procedure may be performed as an independent and sole intervention, specifically to treat a confirmed diagnosis of atrial fibrillation. The procedure may also be performed on a stopped heart, for example, in conjunction with a coronary artery bypass graft (CABG) procedure, or it may be performed on a beating heart.

The described ablation tools and methods of ablation are advantageously applied to form fully transmural myocardial lesions for blocking or preventing symptomatic arrhythmias originating at or propagating from the region of a pulmonary vein. For this purpose, the ablation tip may be formed with the active ablation element shaped in a short arc or segment, and the ablation head may be successively placed at positions to extend a lesion entirely around a single pulmonary vein, or to place a blocking lesion circumscribing a pair of pulmonary veins to build up a complete lesion set. Similarly, the ablation head may be moved to create a different lesion shape.

FIG. 5 schematically illustrates some suitable ablation paths 100, circumscribing plural pulmonary veins at the ostia. Another blocking line (not shown) may be formed, as is known, extending from the ostia to the annulus of the mitral valve. In FIG. 5, heavy dashed lines schematically represent ablation lesions for one lesion set for isolation of the pulmonary veins as described above. This lesion set isolates arrythmogenic foci in the pulmonary veins and it is effective against a common source of atrial fibrillation. Lesion sets of the method may include a set of four circumferential lesions, one around each pulmonary vein, or a set of two circumferential lesions, each encircling a pair of pulmonary veins (e.g., the left pair, or the right pair). The lesions may also include one lesion encircling three pulmonary veins, and a second lesion encircling the remaining vein, or the illustrated lesion set encircling one pair and two separate veins with three circumferential lesions. Preferably, the lesion sets are augmented by the aforesaid additional (endocardial) lesion extending from one of the circumferential lesions to the annulus of the mitral valve, so as to effectively subdivide the atrial wall tissue mass.

As noted above, it is not necessary that all four pulmonary veins be isolated; when a vein is healthy, the physician may apply circumferential lesions only to block the remaining pulmonary veins or sets of veins.

Not only is the construction of the ablation head structure described above well adapted to fit in and around the posterior epicardial surface in region of the pulmonary veins, but the hook, clamp or lasso structures enable the probe to be moved into position and dependably operated to form blocking lesions close to the junction with the atrial wall, in myocardial tissue, to effective block foci within the vein while avoiding stenotic sequelae. The lesion loci reduce size of the continuous connected atrial tissue at the ostia, to more dependably suppress arrythmias. The tip geometry of these probes more effectively accesses and ablates tissue in the region of rough, irregular and varying surface features and tissue thickness at the ostia.

It will be appreciated that the invention provides an improved ablation method and improved ablation devices for practicing the method. Those having ordinary skill in the art will appreciate that various modifications can be made to the described illustrative embodiments and techniques without departing from the scope of the present invention. The invention being thus disclosed, variations and modifications thereof will occur to those having ordinary skill in the art, and such variations and modifications are considered to be within the scope of the invention, as defined by the claims appended hereto and equivalents thereof. All documents, publications and references cited above are hereby expressly incorporated herein by reference in their entirety.

What is claimed is:

1. A method of treating atrial arrythmia, comprising the steps of
accessing the epicardial surface of a heart
positioning a circumferential ablation element epicardially along myocardial tissue at a juncture of a pulmonary vein with the left atrium, and
actuating the ablation element to form a circumferential ablation lesion effective to block signal conduction from the pulmonary vein wall.

2. The method of claim 1, wherein the circumferential ablation element is a lasso element having a deflectable or curved distal end configured to engage the pulmonary vein and positionable at said juncture for forming at least a portion of said circumferential ablation lesion.

3. The method of claim 1, wherein the circumferential ablation element is a clamp or forceps having a pair of members that are movable with respect to each other and are carrying opposed ablation jaws configured to engage a pulmonary vein about at least one circumferential arc along the myocardial tissue.

4. The method of claim 1, wherein the circumferential ablation element is hook element having a substantially rigid ablation element curved to engage a pulmonary vein and positionable at said juncture for forming at least a portion of said circumferential ablation lesion.

5. The method of claim 1, wherein the steps of positioning and actuating the ablation element are repeated one or more times to form contiguous lesions that overlap to form a circumferential lesion blocking conduction between the pulmonary vein, and to form a plurality of circumferential lesions in a lesion set effective to
(i) block conduction between pulmonary vein foci and the atrial wall, and
(ii) diminish mass of a central region of atrial connecting tissue at the juncture such that the central region does not sustain a re-entrant signal.

6. The method of claim 1, wherein the steps of positioning and actuating are repeated one or more times to form contiguous lesions that overlap to form a circumferential lesion about the pulmonary vein, and to form a circumferential lesion blocking conduction from at least one other pulmonary vein or a pair of pulmonary veins.

7. The method of claim 1, wherein the steps of positioning and actuating are repeated one or more times to form contiguous lesions that overlap to form plural circumferential lesions isolating fewer than four pulmonary veins.

8. The method of claim 1, wherein the steps of positioning and actuating are repeated one or more times to form contiguous lesions that overlap to epicardially form plural circumferential lesions extending entirely through myocardial tissue thereby avoiding stenosis of venous tissue while blocking signal conduction between the pulmonary veins and the left atrial wall.

9. An ablation probe, comprising
a handle
an elongated body extending from the handle to a distal end, and
an ablation element at said distal end configured to extend at least partially about a pulmonary vein and form a circumferential blocking lesion through myocardial tissue at the juncture of said pulmonary vein and the left atrium.

10. The ablation probe of claim 9, wherein the ablation element is a lasso element having a deflectable or curved distal end configured to engage the pulmonary vein and positionable at said juncture for forming at least a portion of said circumferential blocking lesion.

11. The ablation probe of claim 9, wherein the ablation probe is a clamping probe having a body carrying opposed ablation jaws and configured to engage the myocardial tissue along at least one arc of the circumferential blocking lesion.

12. The ablation probe of claim 9, wherein the ablation probe includes a hook element curved to engage a pulmonary vein and positionable at said juncture for forming at least a portion of said circumferential blocking lesion.

13. The ablation probe of claim 9, wherein the ablation element is an ablation element selected from among the group of ablation elements having a cryogenic element, an RF ablation element, an ultrasound, microwave, laser, chemical or biological agent delivery, light-activated agent delivery, laser ablation, hot fluid circulating element, and resistance heating ablation element.

14. The ablation probe of claim 9, configured for endoscopic use to access the heart via a port in a closed chest.
